(19) United States
(12) Patent Application Publication
(10) Pub. No.: US 2024/0062858 A1
(43) Pub. Date: Feb. 22, 2024

(54) ELECTRONIC HEALTH RECORDS READER

(71) Applicant: HEALTHPOINTE SOLUTIONS, INC., Austin, TX (US)

(72) Inventors: Nathan Gnanasambandam, Irvine, CA (US); Mark Henry ANDERSON, Newport Coast, CA (US)

(73) Assignee: HEALTHPOINTE SOLUTIONS, INC., Austin, TX (US)

(21) Appl. No.: 18/271,798

(22) PCT Filed: Jan. 11, 2022
(86) PCT No.: PCT/US2022/011930

§ 371 Date: Jul. 11, 2023

Related U.S. Application Data
(60) Provisional application No. 63/135,792, filed on Jan. 11, 2021.

(51) Int. Cl. G16H 10/60; G06F 21/62

(52) U.S. Cl. CPC G16H 10/60; G06F 21/6245

(57) ABSTRACT

A computer-implemented method performed by a cognitive intelligence platform is disclosed herein. The method comprises: receiving, from a plurality of electronic health record systems, medical records associated with medical encounters of a patient with one or more medical providers over a period of time, the medical records including medical information related to care of the patient; analyzing the medical records by classifying the medical information included in the medical records into medical entities, the medical entities including categories having particular shared characteristics related to the care of the patient; generating, based on the medical entities, visual representations of the medical records over the period of time; and providing the visual representations of the medical records to a medical provider.

### CROSS-REFERENCES TO RELATED APPLICATIONS

This application claims priority to U.S. Provisional Application No. 63/135,972 filed Jan. 11, 2021. All applications are hereby incorporated by reference in their entirety for all purposes as if reproduced in full below.

### BACKGROUND

Population health management entails aggregating patient data across multiple health information technology resources, analyzing the data with reference to a single patient, and generating actionable items through which care providers can improve both clinical and financial outcomes. A population health management service seeks to improve the health outcomes of a group by improving clinical outcomes while lowering costs.

### SUMMARY

Representative embodiments set forth herein disclose various techniques for enabling a system and method for operating a clinic viewer on a computing device of a medical personnel.

In one embodiment, a computer-implemented method performed by a cognitive intelligence platform is disclosed. The method comprises: receiving, from a plurality of electronic health record systems, medical records associated with medical encounters of a patient with one or more medical providers over a period of time, the medical records including medical information related to care of the patient; analyzing the medical records by classifying the medical information included in the medical records into medical entities, the medical entities including categories having particular shared characteristics related to the care of the patient; generating, based on the medical entities, visual representations of the medical records over the period of time; and providing the visual representations of the medical records to a medical provider.

In one embodiment, a system, comprises: a memory device containing stored instructions; and a processing device communicatively coupled to the memory device. The processing device executes the stored instructions to: receive, from a plurality of electronic health record systems, medical records associated with medical encounters of a patient with one or more medical providers over a period of time, the medical records including medical information related to care of the patient; analyze the medical records by classifying the medical information included in the medical records into medical entities, the medical entities including categories having particular shared characteristics related to the care of the patient; generate, based on the medical entities, visual representations of the medical records over the period of time; and provide the visual representations of the medical records to a medical provider.

In one embodiment, a computer readable media storing instructions that are executable by a processor to cause a processing device to execute operations comprises: receive, from a plurality of electronic health record systems, medical records associated with medical encounters of a patient with one or more medical providers over a period of time, the medical records including medical information related to care of the patient; analyze the medical records by classifying the medical information included in the medical records into medical entities, the medical entities including categories having particular shared characteristics related to the care of the patient; generate, based on the medical entities, visual representations of the medical records over the period of time; and provide the visual representations of the medical records to a medical provider.

### BRIEF DESCRIPTION OF THE DRAWINGS

For a detailed description of example embodiments, reference will now be made to the accompanying drawings in which:

FIG. 1 shows a block diagram of an example of a health information exchange (HIE) network, in accordance with various embodiments.

FIG. 2 shows a method for analyzing medical records associated with medical encounters of a patient with one or more medical providers, in accordance with various embodiments.

FIG. 3 illustrates an example of medical records including medical information related to care of the patient and the medical information included in the medical records classified into medical entities, in accordance with various embodiments.

FIG. 4 provides an example embodiment of a visual representation that may be generated by cognitive AI engine based on the medical entities, in accordance with various embodiments.

FIG. 5 provides an example embodiment of a visual representation that may be generated by cognitive AI engine based on the medical entities, in accordance with various embodiments.

FIG. 6 provides an example embodiment of a visual representation that may be generated by cognitive AI engine based on the medical entities, in accordance with various embodiments.

FIG. 7 shows a method for identifying a negation cue modifying a piece of text in the medical information and determining a probability of existence of the aspect of care of the patient, in accordance with various embodiments.

FIG. 8 shows a method for determining an effectiveness of a treatment plan received by the patient, in accordance with various embodiments.

FIG. 9 shows a method for identifying a negation cue modifying a piece of text in the medical information, in accordance with various embodiments.

FIG. 10 illustrates a detailed view of a computing device that can represent any of the computing devices of FIG. 1 used to implement the various platforms and techniques described herein, according to some embodiments.

### NOTATION AND NOMENCLATURE

Various terms are used to refer to particular system components. Different companies may refer to a component by different names—this document does not intend to distinguish between components that differ in name but not function. In the following discussion and in the claims, the terms “including” and “comprising” are used in an open-ended fashion, and thus should be interpreted to mean “including, but not limited to . . . .” Also, the term “couple” or “couples” is intended to mean either an indirect or direct connection. Thus, if a first device couples to a second device, that connection may be through a direct connection or through an indirect connection via other devices and connections.

The terminology used herein is for the purpose of describing particular example embodiments only, and is not intended to be limiting. As used herein, the singular forms “a,” “an,” and “the” may be intended to include the plural forms as well, unless the context clearly indicates otherwise. The method steps, processes, and operations described herein are not to be construed as necessarily requiring their performance in the particular order discussed or illustrated, unless specifically identified as an order of performance. It is also to be understood that additional or alternative steps may be employed.

The terms first, second, third, etc. may be used herein to describe various elements, components, regions, layers and/or sections; however, these elements, components, regions, layers and/or sections should not be limited by these terms. These terms may be only used to distinguish one element, component, region, layer, or section from another region, layer, or section. Terms such as “first,” “second,” and other numerical terms, when used herein, do not imply a sequence or order unless clearly indicated by the context. Thus, a first element, component, region, layer, or section discussed below could be termed a second element, component, region, layer, or section without departing from the teachings of the example embodiments. The phrase “at least one of,” when used with a list of items, means that different combinations of one or more of the listed items may be used, and only one item in the list may be needed. For example, “at least one of: A, B, and C” includes any of the following combinations: A, B, C, A and B, A and C, B and C, and A and B and C. In another example, the phrase “one or more” when used with a list of items means there may be one item or any suitable number of items exceeding one.

Some embodiments are described in connection with thresholds. As used herein, satisfying a threshold may refer to a value being greater than the threshold, more than the threshold, higher than the threshold, greater than or equal to the threshold, less than the threshold, fewer than the threshold, lower than the threshold, less than or equal to the threshold, equal to the threshold, or the like.

### DETAILED DESCRIPTION

The following discussion is directed to various embodiments. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

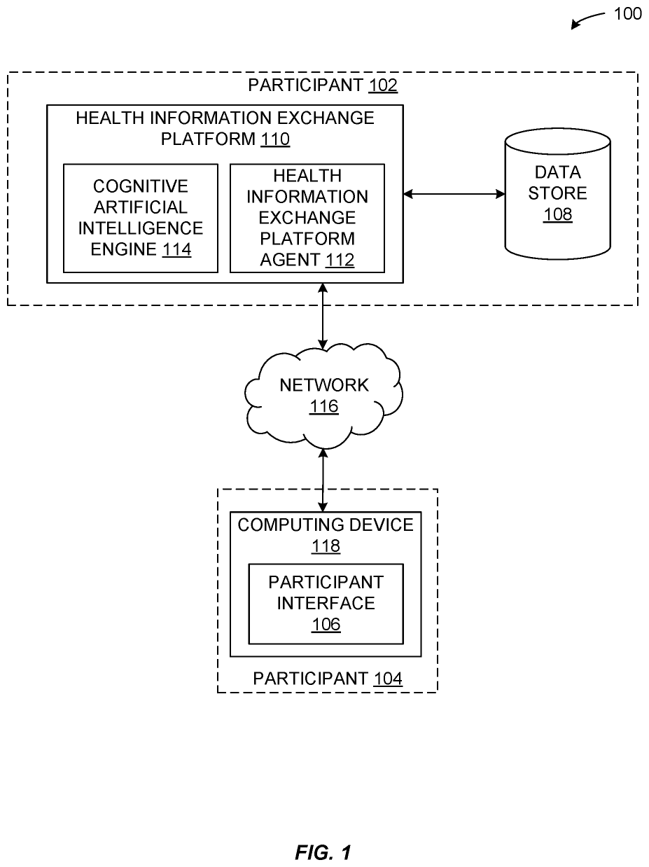

A method and a system for analyzing medical records associated with medical encounters of a patient with one or more medical providers are disclosed herein. FIG. 1 shows a block diagram of an example of a health information exchange (HIE) network 100 that enables an exchange of health information between participants in HIE network 100, in accordance with various embodiments described herein. HIE network 100 allows doctors, nurses, pharmacists, other health care providers, and patients to appropriately access and securely share medical information of a patient electronically. As shown in FIG. 1, HIE network 100 includes participants 102 and 104. For illustration purposes, HIE network 100 is shown to have only participants 102 and 104 but may include any number of participants. Participants may represent electronic health record (EHR) systems that are associated with any type of health care provider or may be a patient. EHR systems as used herein refers to network-connected, enterprise-wide information systems or other information networks of a health provider. In some embodiments, each participant in HIE network 100 may be associated with a different, disparate health provider. A health care provider as used herein refers to entities that provide health services to patients such as (but not limited to) hospitals, doctor offices, laboratories, specialists, medical imaging facilities, pharmacies, emergency facilities, and school and workplace clinics. The health information exchanged between participants in HIE network 100 may include health records associated with a patient such as medical and treatment histories of patients but can go beyond standard clinical data collected by a doctor's office/health provider. For example, health records may include a patient's medical history, diagnoses, medications, treatment plans, immunization dates, allergies, radiology images, and laboratory and test results.

More specifically, FIG. 1 illustrates a high-level overview of a HIE platform 110 that enables participant 102 to securely share medical information with participant 104. HIE platform 110 may be a component of network-connected, enterprise-wide information systems or other information networks maintained by participant 102. As further shown in FIG. 1, HIE platform 110 includes a HIE platform agent 112 and a cognitive artificial intelligence (AI) engine 114. For purposes of this discussion, the HIE platform 110 provides services in the health industry, thus the examples discussed herein are associated with the health industry. However, any service industry can benefit from the disclosure herein, and thus the examples associated with the health industry are not meant to be limiting.

HIE platform 110 includes several computing devices, where each computing device, respectively, includes at least one processor, at least one memory, and at least one storage (e.g., a hard drive, a solid-state storage device, a mass storage device, and a remote storage device). The individual computing devices can represent any form of a computing device such as a desktop computing device, a rack-mounted computing device, and a server device. The foregoing example computing devices are not meant to be limiting. On the contrary, individual computing devices implementing HIE platform 110 can represent any form of computing device without departing from the scope of this disclosure.

In various embodiments, the several computing devices executing within HIE platform 110 are communicably coupled by way of a network/bus interface. Furthermore, HIE platform agent 112 and a cognitive AI engine 114 may be communicably coupled by one or more inter-host communication protocols. In some embodiments, HIE platform agent 112 and a cognitive AI engine 114 may execute on separate computing devices. Still yet, in some embodiments, HIE platform agent 112 and a cognitive AI engine 114 may be implemented on the same computing device or partially on the same computing device, without departing from the scope of this disclosure.

The several computing devices work in conjunction to implement components of HIE platform 110 including HIE platform agent 112 and cognitive AI engine 114. HIE platform 110 is not limited to implementing only these components, or in the manner described in FIG. 1. That is, HIE platform 110 can be implemented, with different or additional components, without departing from the scope of this disclosure. The example HIE platform 110 illustrates one way to implement the methods and techniques described herein.

In FIG. 1, HIE platform agent 112 represents a set of instructions executing within HIE platform 110 that implement a client-facing component of HIE platform 110. HIE platform agent 112 may be configured to enable interaction between participant 102 and participant 104. Various user interfaces may be provided to computing devices communicating with HIE platform agent 112 executing in HIE platform 110. For example, a participant interface 106 may be presented in a standalone application executing on a computing device 118 or in a web browser as website pages. In some embodiments, HIE platform agent 110 may be installed on computing device 118 of participant 104. In some embodiments, computing device 118 of participant 104 may communicate with HIE platform 110 in a client-server architecture. In some embodiments, HIE platform agent 112 may be implemented as computer instructions as an application programming interface.

Computing device 118 represents any form of a computing device, or network of computing devices, e.g., a personal computing device, a smart phone, a tablet, a wearable computing device, a notebook computer, a media player device, and a desktop computing device. Computing device 118 includes a processor, at least one memory, and at least one storage. In some embodiments, an employee or representative of participant 104 may use participant interface 106 to input a given text posed in natural language (e.g., typed on a physical keyboard, spoken into a microphone, typed on a touch screen, or combinations thereof) and interact with HIE platform 110, by way of HIE platform agent 112.

The HIE network 100 includes a network 116 that communicatively couples various devices, including HIE platform 110 and computing device 118. The network 116 can include local area network (LAN) and wide area networks (WAN). The network 116 can include wired technologies (e.g., Ethernet C)) and wireless technologies (e.g., Wi-Fi®, code division multiple access (CDMA), global system for mobile (GSM), universal mobile telephone service (UMTS), Bluetooth®, and ZigBee®. For example, computing device 118 can use a wired connection or a wireless technology (e.g., Wi-Fi®) to transmit and receive data over network 116.

With continued reference to FIG. 1, cognitive AI engine 114 represents a set of instructions executing within HIE platform 110 that is configured to collect, analyze, and process health information data associated with a patient from various sources and entities. Assume for the sake of illustration participant 104 is a primary care provider for a patient. Throughout the course of a relationship between participant 104 and the patient, participant 104 may collect and generate health information data associated with a patient (such as any diagnoses, prescriptions, treatment plans, etc.). Each medical encounter may be included in the health information data. Medical encounter as used herein refers to an interaction between a patient and a healthcare provider for the purpose of providing healthcare services or assessing the health status of a patient. For example, medical encounters may include inpatient, outpatient, emergency, and telehealth interactions between the patient and the health provider. In some embodiments, an employee of participant 104, using computing device 118, may provide the data associated with the patient to HIE platform 110.

Cognitive AI engine 114 may also collect health information data from other participants in HIE network 100. For example, HIE platform 110 may receive secure health information electronically from another care provider to support coordinated care between participant 104 and the other provider. As another example, HIE platform 110 may receive a request for health information from another participant and cognitive AI engine 114 may collect information associated with the request for health information. For example, the collected information associated with requests for health information may include identifying information associated with the requesting participant (e.g., national provider identifier number, name of requesting medical professional, etc.), location of the participant, types of health information requested (e.g., prescription information, patient demographics, patient conditions, etc.), and date and time of the request.

Further, cognitive AI engine 114 may use natural language processing (NLP), data mining, and pattern recognition technologies to process the retrieved medical information. More specifically, cognitive AI engine 204 may use different AI technologies to understand language, translate content between languages, recognize elements in images and speech, and perform sentiment analysis. For example, cognitive AI engine 114 may use natural language processing (NLP) and data mining and pattern recognition technologies to collect and process information provided in different health information resources. For example, cognitive AI engine 114 may use NLP to extract and interpret hand written notes and text (e.g., a doctor's notes). As another example, cognitive AI engine 114 may use imaging extraction techniques, such as optical character recognition (OCR) and/or use a machine learning model trained to identify and extract certain health information. OCR refers to electronic conversion of an image of printed text into machine-encoded text and may be used to digitize health information. As another example, pattern recognition and/or computer vision may also be used to extract information from health information resources. Computer vision may involve image understanding by processing symbolic information from image data using models constructed with the aid of geometry, physics, statistics, and/or learning theory. Pattern recognition may refer to electronic discovery of regularities in data through the use of computer algorithms and with the use of these regularities to take actions such as classifying the data into different categories and/or determining what the symbols represent in the image (e.g., words, sentences, names, numbers, identifiers, etc.). Finally, cognitive AI engine 114 may use NLU techniques to process unstructured data using text analytics to extract entities, relationships, keywords, semantic roles, and so forth.

In some embodiments, cognitive AI engine 114 may use the same technologies to synthesize data from various information sources and entities, while weighing context and conflicting evidence. Still yet, in some embodiments, cognitive AI engine 114 may use one or more machine learning models. The one or more machine learning models may be generated by a training engine and may be implemented in computer instructions that are executable by one or more processing devices of the training engine, the cognitive AI engine 114, another server, and/or the computing device 118. To generate the one or more machine learning models, the training engine may train, test, and validate the one or more machine learning models. The training engine may be a rackmount server, a router computer, a personal computer, a portable digital assistant, a smartphone, a laptop computer, a tablet computer, a camera, a video camera, a netbook, a desktop computer, a media center, or any combination of the above. The one or more machine learning models may refer to model artifacts that are created by the training engine using training data that includes training inputs and corresponding target outputs. The training engine may find patterns in the training data that map the training input to the target output, and generate the machine learning models that capture these patterns.

For example, the one or more machine learning models may be trained to analyze the medical records by classifying the medical information included in the medical records into medical entities. The medical entities may include categories having particular shared characteristics related to the care of the patient. For example, medical entities may include: conditions, medications, laboratory results, test results, procedures, symptoms, and life style choices. To help further illustrate, in some embodiments, one or more machine learning algorithms may be used to classify the medical information included in the medical records into medical entities. As described, cognitive AI engine 114 may include a machine learning (ML) model generator and one or more ML models. The ML model generator may be configured to generate ML models to analyze the medical records. The ML models may be deployed in cognitive AI engine 114.

In an embodiment, the ML model generator may be configured to generate a model used to classify the medical information included in the medical records into medical entities. For example, the ML model generator may include a machine learning algorithm. The machine learning algorithm may be provided medical information of other patients and medical entities corresponding to the medical information. The machine learning algorithm may be executed by the model generator to generate the model based on the medical information of other patients and medical entities corresponding to the medical information. The model may use a training dataset (e.g., medical information of other patients and medical entities corresponding to the medical information) and calculate how to best map examples of input medical information to the medical entities.

The ML model generator may also include a machine learning (ML) application that implements the ML algorithm to generate the model. The model may be generated using any suitable techniques, including supervised machine learning model generation algorithms such as supervised vector machines (SVM), linear regression, logistic regression, naïve Bayes, linear discriminant analysis, decision trees, k-nearest neighbor algorithm, neural networks, recurrent neural network, etc. In some embodiments, unsupervised learning algorithms may be used such as clustering or neural networks.

Note that the model may be generated in various forms. In accordance with one embodiment, the model may be generated according to a suitable machine-learning algorithm mentioned elsewhere herein or otherwise known. In an embodiment, the ML model generator may implement a gradient boosted tree algorithm or other decision tree algorithm to generate and/or train the model in the form of a decision tree. The decision tree may be traversed with input data (medical information of a patient, etc.) to identify one or more medical entities that the medical information maps to.

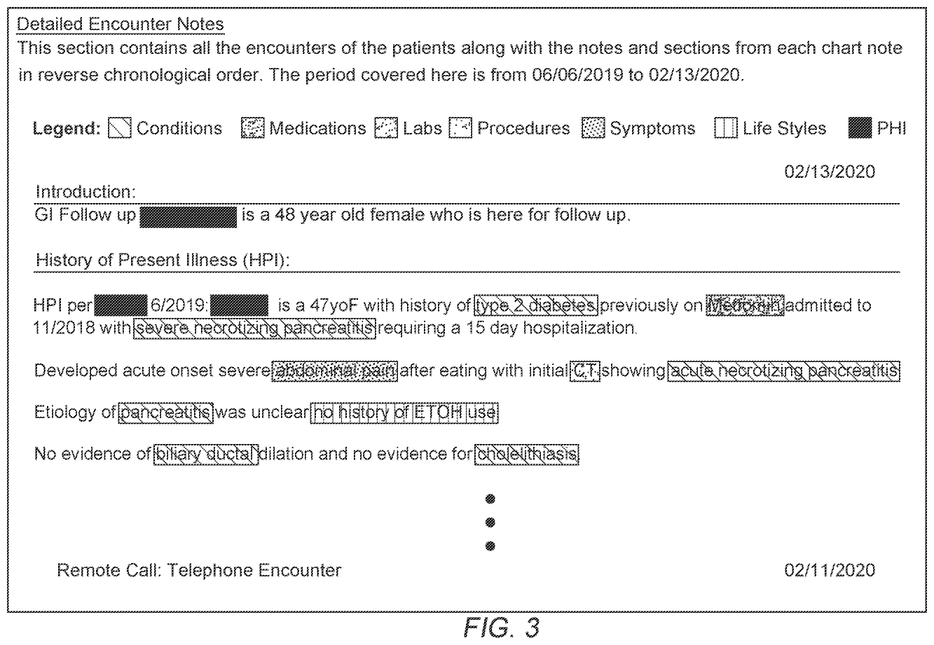

For example, FIG. 3 illustrates an example user interface including medical records including medical information related to care of the patient and the medical information included in the medical records classified into medical entities. In FIG. 3, medical information classified into various medical entities indicated in the legend is depicted (i.e., conditions, medications, laboratory results, test results, procedures, symptoms, and life style choices). For example, as shown in FIG. 3, medical information, such as type 2 diabetes and necrotizing pancreatitis, is identified as conditions. As another example, medical information, such as Metformin, is identified as medication. In some embodiments, in FIG. 3, cognitive AI engine 114 may redact personally identifiable information (e.g., a patient's name, a doctor's name, etc.,) from the medical records. As shown in FIG. 3, personally identifiable information included in the medical record may be redacted and indicated as protected health information (PHI). PHI may include any information about health status, provision of health care, or payment for health care that is created or collected and can be linked to a specific individual. As another example, in FIG. 3, medical information, such as “no history of ETOH use,” is identified as life style. Life style practices may indicate the way in which a person lives and may include practices related to nutrition, sleep, exercise, smoking, consumption of alcohol, etc.

Additionally, with continued reference to the example above, clinical-based evidence, clinical trials, physician research, and the like that includes various information pertaining to different medical information may be input as training data to the one or more machine learning models. The information may pertain to facts, properties, attributes, concepts, conclusions, risks, correlations, complications, etc. of the medical conditions. Keywords, phrases, sentences, cardinals, numbers, values, objectives, nouns, verbs, concepts, and so forth may be specified (e.g., labeled) in the information such that the machine learning models learn which ones are associated with the medical conditions. The information may specify predicates that correlates the information in a logical structure such that the machine learning models learn the logical structure associated with the medical information. Other sources including information pertaining to other types of health information (e.g., patient demographics, patient history, medications, allergies, procedures, diagnosis, lab results, immunizations, etc.) may input as training data to the one or more machine learning models.

Cognitive AI engine 114 can be configured to train the ML models based on medical information associated with participants. Additionally, cognitive AI engine 114 can be configured to update the ML models based on medical information. For example, cognitive AI engine 114 may maintain the ML models by continuously retraining the ML models based on medical information and medical entities corresponding to the medical information. For example, the medical provider may be a physician that performed a medical test on the participant and the medical information may include the type of medical test and the result of the medical test, among other information. In some embodiments, the medical information may include information pertaining to a medical test performed for the patient, a medical metric pertaining to the patient, a result of the medical test performed for the patient, a license of the medical personnel, a degree of the medical personnel, a timestamp of the medical information, or some combination thereof. Further, cognitive AI engine 114 may apply the medical information to one or more ML models, classify the medical information into medical entities, and update the one or more ML models based on the classifications of the medical entities. In some embodiments, the one or more machine learning models may be stored in a data store 108.

As depicted, the user interface provided by the HIE platform 110 a legend may include graphical user elements that represent various medical information, such as conditions, medications, lab results, symptoms, life style choices, etc. The AI engine 114 may process medical records to identify the medical information in the medical record and may use the graphical user elements to tag the medical information in the medical records. Such tagging may provide more efficient parsing and analysis of the medical records by “highlighting” respective medical information in the medical records. As a result, the disclosed techniques may provide an enhanced user interface that enhances a user's experience of using the computing device because the pertinent information is called out in the user interface using the graphical user elements and the user does not have to read the entire medical record to determine the salient medical information.

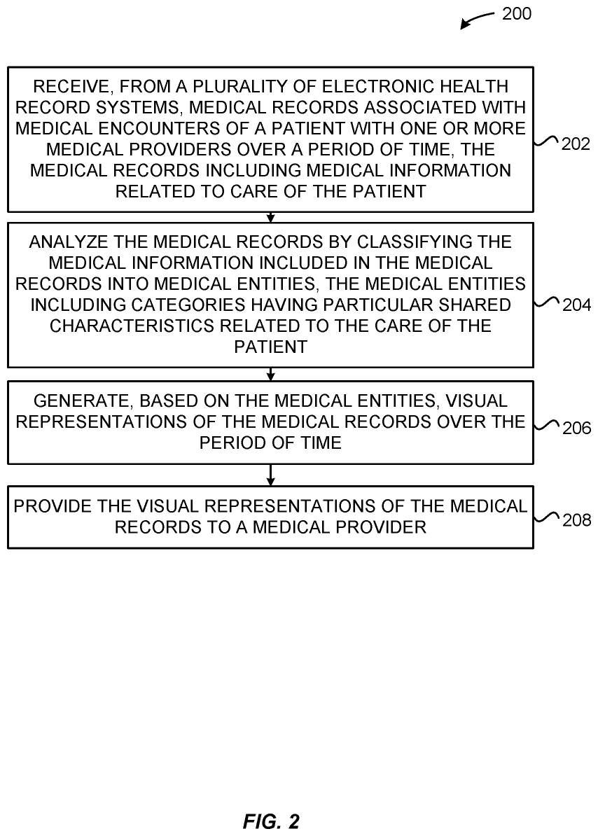

To explore the foregoing in further detail, FIG. 2 will now be described. FIG. 2 shows a method 200 for analyzing medical records associated with medical encounters of a patient with one or more medical providers. As shown in FIG. 2, method 200 beings at step 202. At step 202, medical records are received from a plurality of electronic health record systems, where the medical records are associated with medical encounters of a patient with one or more medical providers over a period of time and include medical information related to care of the patient. For example, with reference to FIG. 1, cognitive AI engine 114 may receive medical records from a plurality of electronic health record systems (e.g., participant 104) via network 116.

At step 204, the medical records are analyzed by classifying the medical information included in the medical records into medical entities, where the medical entities included categories having particular shared characteristics related to the care of the patient. For example, as described with reference to FIG. 1, cognitive AI engine 114 classify the medical information included in the medical records into medical entities. To help further illustrate, in FIG. 3, medical information classified into various medical entities indicated in the legend is depicted (i.e., conditions, medications, laboratory results, test results, procedures, symptoms, and life style choices). For example, as shown in FIG. 3, medical information, such as type 2 diabetes and necrotizing pancreatitis, is identified as conditions. As another example, medical information, such as Metformin, is identified as medication.

To help further illustrate, cognitive AI engine 114 is configured to apply the medical information to the model. More specifically, cognitive AI engine 114 provides the medical information to the model and cognitive AI engine 114 receives, from the employee-employer compatibility model, an indication including the one or more medical entities that the medical information corresponds to.

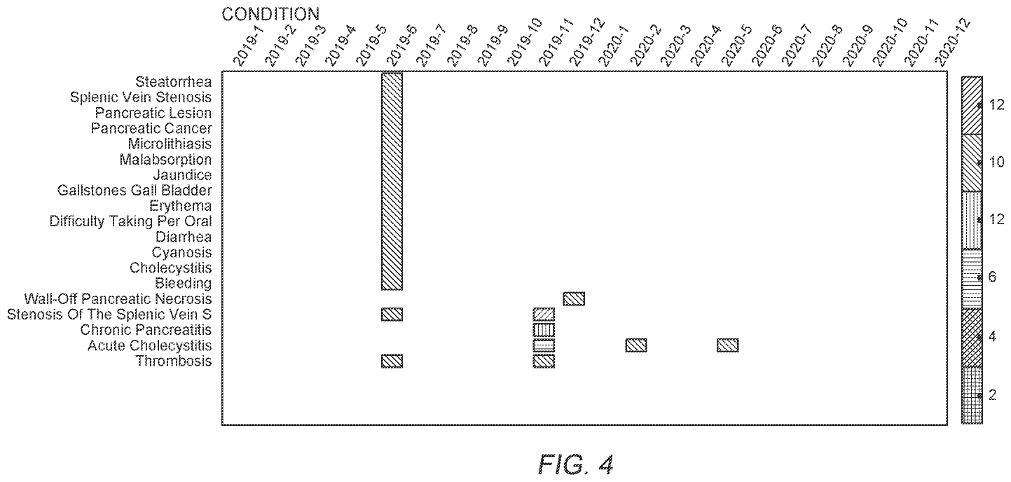

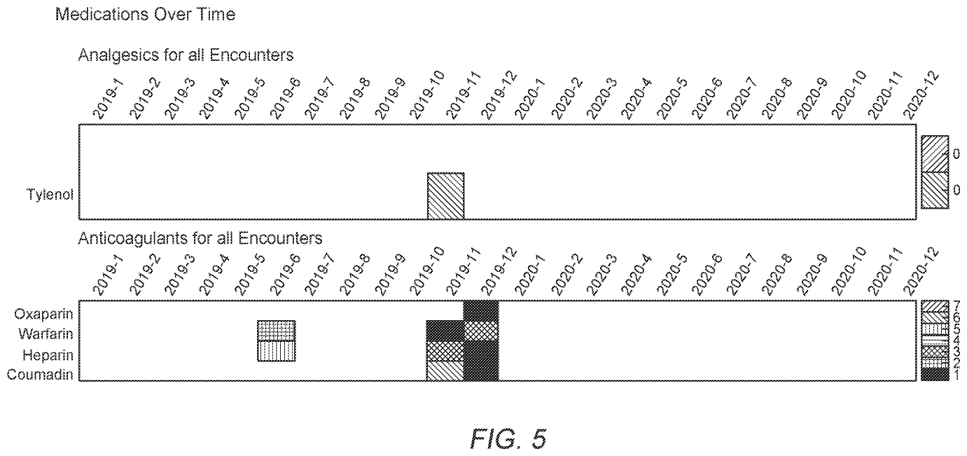

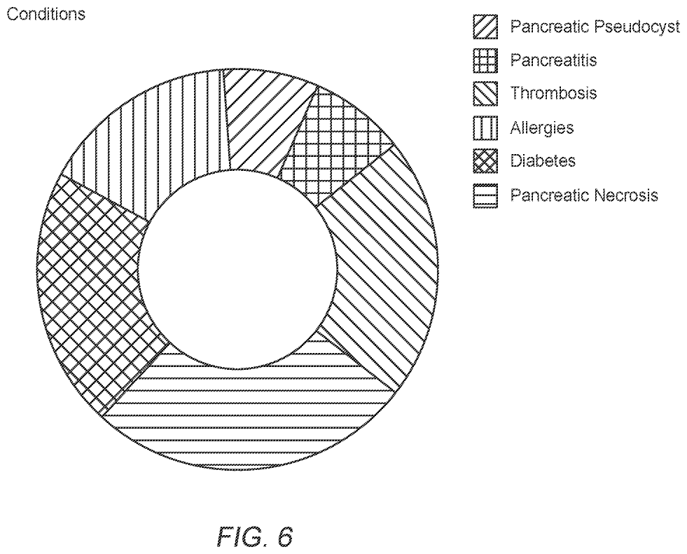

At step 206, based on the medical entities, visual representations of the medical records over the period of time are generated. For example, with continued reference to FIG. 1, cognitive AI engine 114 may generate, based on the medical entities, visual representations of the medical records. The visual representations may provide a medical provider with an accurate and easily consumed depiction of a patient's health and healthcare coverage that the patient received over a period of time (e.g., a year). FIGS. 4-6 provide exemplary embodiments of visual representations that may be generated by cognitive AI engine 114 based on the medical entities. For example, FIG. 4 depicts chronologically a number of occurrences of medical information identified as conditions that are in a patient's medical records. As shown in FIG. 4, the condition, acute cholecystitis, is identified as being mentioned in the patient's medical records for three different medical encounters. As another example, FIG. 5 depicts chronologically a number of occurrences of medical information identified as medication that are in a patient's medical records. This enables the identification of when conditions or medications are documented and if they are documented consistently. Still yet, as another example, FIG. 6 provides another example of depicting a number of occurrences of medical information identified as conditions that are in a patient's medical records. However, FIG. 6 easily allows a medical provider to see which medical conditions have been included most in the patient's medical records.

At step 208, the visual representations of the medical records are provided to a medical provider. For example, with continued reference to FIG. 1, cognitive AI engine 114 may provide, via network 116, the visual representations of the medical records to a medical provider. A medical provider may view the visual representations via participant interface 106. In some embodiments, participant interface 106 may be presented in a standalone application executing on a computing device 118 or in a web browser as website pages. An employee or representative of participant 104 may interact with HIE platform 110 using participant interface 106. For example, the employee or representative of participant 104 may request health information associated with a patient (e.g., through utterances of one or more words, typing of a request, or uploading of an image), and participant interface 106 may capture user input representing a request of the patient from the interaction and provide the user input to HIE platform 110. HIE platform agent 112 may be configured to provide the visual representations to client computing device 118.

In some embodiments, cognitive AI engine 114 may generate the visual representation by: generating graphical user interface elements to represent the visual representations of the medical records over the period of time; and causing the graphical user interface elements to be presented on a single user interface generated by the cognitive intelligence platform.

In some embodiments, cognitive AI engine 114 may receive a user-generated natural language query from a user interface (e.g., participant interface 106) associated with the medical provider and generate, based on the medical information, a response to the user-generated natural language query. For example, a medical provider associated with participant 104 may inquire about symptoms associated with a condition that a patient may be experiencing. Cognitive AI engine 114 may provide a response to the participant interface 106 associated with the medical provider. The response may include any symptoms associated with a disease that the patient is experiencing and not experiencing that are mentioned in the patient's medical records.

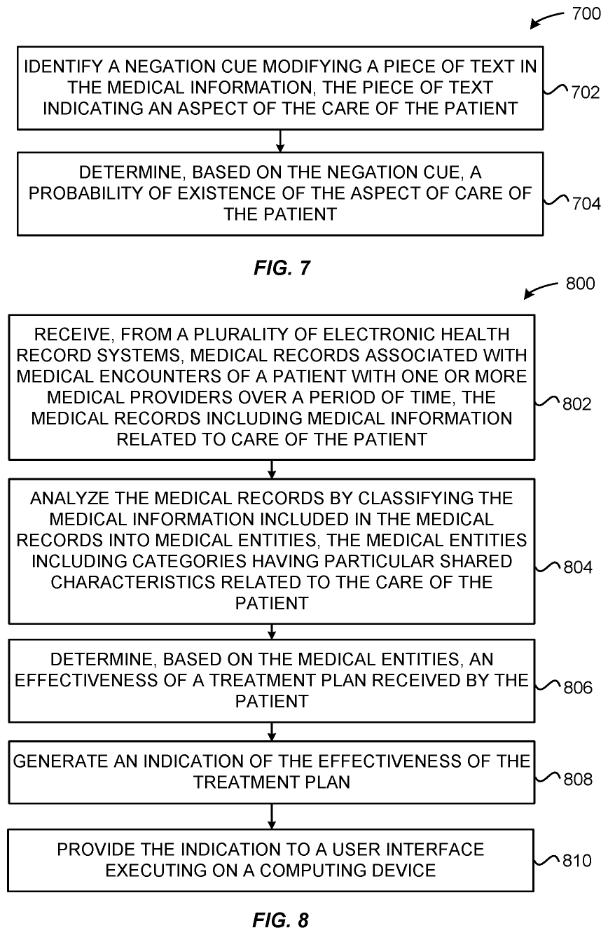

To explore the foregoing in further detail, FIG. 7 will now be described. FIG. 7 shows a method 700 for identifying a negation cue modifying a piece of text in the medical information and determining a probability of existence of the aspect of care of the patient. As shown in FIG. 7, method 700 beings at step 702. At step 702, a negation cue modifying a piece of text in the medical information is identified, where the piece of text indicates an aspect of the care of the patient. For example, with reference to FIG. 1, cognitive AI engine 114 may parse a medical record and using machine learning methods, such as NLP, identify a negation cue (e.g., “never”, “not”, “no longer”, “absence”, etc.) modifying a piece of text in the medical record.

At step 704, based on the negation cue, a probability of existence of the aspect of care of the patient is determined. For example, with reference to FIG. 1, cognitive AI engine 114 may determine, based on the negation cue, a probability of existence of the aspect of care of the patient. After identification of a negation cue in the medical information of medical records associated with a patient, cognitive AI engine 114, using machine learning methods, such as NLP, may determine the contextual relationship between the negation cue and words immediately preceding and proceeding the negation cue. Based on the relationship of the negation cue and surrounding text, cognitive AI engine 114 may determine a probability of existence of the aspect of care of the patient. For example, medical records of a patient may indicate that a patient does not have certain symptoms (e.g., increased thirst, frequent urination, extreme hunger, unexplained weight loss, presence of ketones in the urine, etc.) of diabetes and based on this determination, cognitive AI engine 114 may determine a probability that the patient has diabetes. Further, cognitive AI engine 114 may determine a probability of existence of condition in a patient by weighing symptoms that a patient has experienced against symptoms the patient is not experiencing. Moreover, in some embodiments, cognitive AI engine 114 may eliminate, based on the medical information (e.g., symptoms, test results, etc.), one or more potential diagnoses from a list of possible conditions the patient may potentially have. Further, in some embodiment, cognitive AI engine 114, using machine learning methods, may classify one or more aspects of the medical information included in the medical records into any of the following: definite negations, definite preferences, definite rule outs, probable negations, probable preferences, and probable rule outs. Definite negations and definite preferences are medical information that is comprehended without ambiguity or uncertainty. Similarly, definite rule out includes an elimination or exclusion of something (e.g., a condition, diagnosis, etc.) from consideration without ambiguity or uncertainty. In contrast, probable negations, probable preferences, and probable rule outs represent medical information that is likely (rather than definitely) to be experienced by the patient or not experienced by the patient.

To explore the foregoing in further detail, FIG. 8 will now be described. FIG. 8 shows a method 800 for determining an effectiveness of a treatment plan received by the patient. As shown in FIG. 8, method 800 beings at step 802. At step 802, medical records are received from a plurality of electronic health record systems, where the medical records are associated with medical encounters of a patient with one or more medical providers over a period of time and include medical information related to care of the patient. For example, with reference to FIG. 1, cognitive AI engine 114 may receive medical records from a plurality of electronic health record systems (e.g., participant 104) via network 116.

At step 804, the medical records are analyzed by classifying the medical information included in the medical records into medical entities, where the medical entities include categories having particular shared characteristics related to the care of the patient. For example, as described with reference to FIGS. 1-3, cognitive AI engine 114 may classify the medical information included in the medical records into medical entities.

At step 806, based on the medical entities, an effectiveness of a treatment plan received by the patient is determined. For example, with continued reference to FIG. 1, cognitive AI engine 114 may determine, based on the medical entities, an effectiveness of a treatment plan received by the patient. In some embodiments, cognitive AI engine 114 may determine the effectiveness of the treatment plan by analyzing a patient health outcome in response to medical care provided to the patient, where the medical care is specified in the treatment plan. A patient health outcome as used herein refers to changes in health status, usually due to a medical intervention. For example, achieving a good patient health outcome due to the treatment plan may be a factor in determining the effectiveness of a treatment plan received by the patient. In some embodiments, cognitive AI engine 114 may determine the effectiveness of the treatment plan by analyzing any of the following: adequacy of facilities of where the treatment plan is provided, adequacy of equipment administering the treatment plan, qualifications of medical staff administering the treatment plan, and qualifications of an organization administering the treatment plan. Any of these items may be a factor in determining the effectiveness of a treatment plan received by the patient. Moreover, in some embodiments, cognitive AI engine 114 may determine the effectiveness of the treatment plan by examining a process of care provided to the patient based on at least one of appropriateness, completeness, and redundancy of the medical information related to the treatment plan included in the medical records. Any of these items may be a factor in determining the effectiveness of a treatment plan received by the patient.

In some embodiments, cognitive AI engine 114 is configured to train, based on medical information associated with a plurality of patients, a medical treatment efficacy prediction model. For example, the training data may include the analysis of effectiveness of treatment plans for other patients. The analysis may include factors that were associated with application of the treatment plan and a corresponding indication as to how effective the treatment plan was. The medical treatment efficacy prediction model may define associations between the factors described above and the effectiveness of a treatment plan. Further, cognitive AI engine 114 may apply the treatment plan associated with the patient to the medical treatment efficacy prediction model and the factors described above and receive, from the medical treatment efficacy prediction model, an indication that the treatment plan meets a threshold probability of being an effective treatment plan.

At step 808, an indication of the effectiveness of the treatment plan is generated. For example, with continued reference to FIG. 1, cognitive AI engine 114 may generate an indication of the effectiveness of the treatment plan. In some embodiments, cognitive AI engine 114 may generate, based on the determination at step 806, the indication of the effectiveness of the treatment plan. The indication may include an effectiveness score and evidence (e.g., details on the process of care or outcome of the treatment plan) supporting whether or not the treatment plan was effective.

At step 810, the indication is provided to a user interface executing on a computing device. For example, with continued reference to FIG. 1, cognitive AI engine 114 may provide, via network 116, the indication to a medical provider. A medical provider may view the indication via participant interface 106.

In some embodiments, cognitive AI engine 114 may generate the indication by: generating graphical user interface elements to represent the indication of the effectiveness of the treatment plan; and causing the graphical user interface elements to be presented on a single user interface generated by the cognitive intelligence platform.

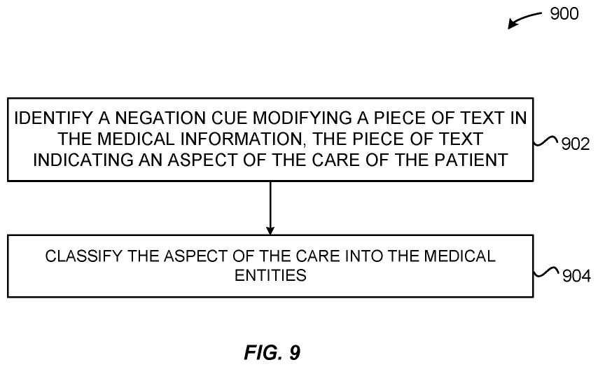

To explore the foregoing in further detail, FIG. 9 will now be described. FIG. 9 shows a method 900 for identifying a negation cue modifying a piece of text in the medical information, where the piece of text indicates an aspect of the care of the patient and classifies the aspect of the care into the medical entities. As shown in FIG. 9, method 900 beings at step 902. At step 902, a negation cue modifying a piece of text in the medical information is identified, where the piece of text indicates an aspect of the care of the patient. For example, with reference to FIG. 1, cognitive AI engine 114 may parse a medical record and using machine learning methods, such as NLP, identify a negation cue (e.g., “never”, “not”, “no longer”, “absence”, etc.) modifying a piece of text in the medical record.

At step 904, the aspect of the care is classified into the medical entities. For example, with reference to FIGS. 1-3, cognitive AI engine 114 may classify the aspect of the care into the medical entities. The process described above with reference to FIGS. 1-3 may also be implemented to perform step 904.

There are several technical benefits for analyzing medical records as described above. One such benefit is providing the visual representations of lengthy medical records in a concise and comprehendible format to a medical provider. This prevents the medical provider from seeking medical information by having to scroll through pages of medical records. Each scroll is a request to the network and by reducing the chance that the user will make that call for additional medical information, computing resources are saved (e.g., processing, network, memory). Also, the user interface includes the most relevant medical information, thereby providing an improved user interface that may increase the user's experience using the computing device and platform by not having to perform a lot of individual searches. In addition, computing resources are further saved by employing AI technologies to process large amounts of data to better curate medical information for a medical provider.

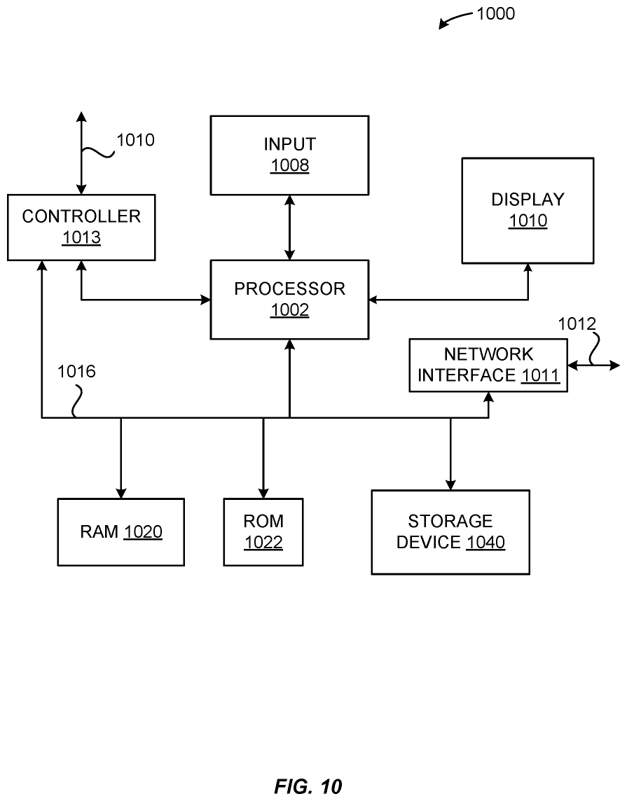

FIG. 10 illustrates a detailed view of a computing device 1000 that can be used to implement the various components described herein, according to some embodiments. In particular, the detailed view illustrates various components that can be included in the computing device 118 illustrated in FIG. 1, as well as the several computing devices implementing health information exchange platform 110. As shown in FIG. 10, computing device 1000 can include a processor 1002 that represents a microprocessor or controller for controlling the overall operation of computing device 1000. Computing device 1000 can also include a user input device 1008 that allows a user of computing device 1000 to interact with computing device 1000. For example, the user input device 1408 can take a variety of forms, such as a button, keypad, dial, touch screen, audio input interface, visual/image capture input interface, input in the form of sensor data, and so on. Still further, computing device 1000 can include a display 1010 that can be controlled by the processor 1002 to display information to the user. A data bus 1016 can facilitate data transfer between at least a storage device 1040, processor 1002, and a controller 1013. Controller 1013 can be used to interface with and control different equipment through an equipment control bus 1014. Computing device 1000 can also include a network/bus interface 1011 that couples to a data link 1012. In the case of a wireless connection, network/bus interface 1011 can include a wireless transceiver.

As noted above, computing device 1000 also includes storage device 1040, which can comprise a single disk or a collection of disks (e.g., hard drives), and includes a storage management module that manages one or more partitions within storage device 1040. In some embodiments, storage device 1040 can include flash memory, semiconductor (solid-state) memory or the like. Computing device 1000 can also include a Random-Access Memory (RAM) 1020 and a Read-Only Memory (ROM) 1022. ROM 1022 can store programs, utilities or processes to be executed in a non-volatile manner. RAM 1020 can provide volatile data storage, and stores instructions related to the operation of processes and applications executing on the computing device.

The various aspects, embodiments, implementations or features of the described embodiments can be used separately or in any combination. Various aspects of the described embodiments can be implemented by software, hardware or a combination of hardware and software. The described embodiments can also be embodied as computer readable code on a computer readable medium. The computer readable medium is any data storage device that can store data which can thereafter be read by a computer system. Examples of the computer readable medium include read-only memory, random-access memory, CD-ROMs, DVDs, magnetic tape, hard disk drives, solid-state drives, and optical data storage devices. The computer readable medium can also be distributed over network-coupled computer systems so that the computer readable code is stored and executed in a distributed fashion.

Consistent with the above disclosure, the examples of systems and method enumerated in the following clauses are specifically contemplated and are intended as a non-limiting set of examples.
Clause 1. A computer-implemented method performed by a cognitive intelligence platform, the method comprising: receiving, from a plurality of electronic health record systems, medical records associated with medical encounters of a patient with one or more medical providers over a period of time, the medical records including medical information related to care of the patient; analyzing the medical records by classifying the medical information included in the medical records into medical entities, the medical entities including categories having particular shared characteristics related to the care of the patient; generating, based on the medical entities, visual representations of the medical records over the period of time; and providing the visual representations of the medical records to a medical provider.
Clause 2. The computer-implemented method, the method further comprising: redacting personally identifiable information from the medical records.
Clause 3. The computer-implemented method, wherein analyzing the medical records further comprises: identifying a negation cue modifying a piece of text in the medical information, the piece of text indicating an aspect of the care of the patient; and determining, based on the negation cue, a probability of existence of the aspect of care of the patient.
Clause 4. The computer-implemented method, wherein analyzing the medical records further comprises: classifying one or more aspects of the medical information included in the medical records into any of the following: definite negations, definite preferences, definite rule outs, probable negations, probable preferences, and probable rule outs.
Clause 5. The computer-implemented method, wherein the medical entities includes at least one of the following: conditions, medications, laboratory results, test results, procedures, symptoms, and life style choices.
Clause 6. The computer-implemented method, the method further comprising: eliminating, based on the medical information, one or more potential diagnoses from a list of possible conditions the patient may have.
Clause 7. The computer-implemented method, the method further comprising: receiving a user-generated natural language query from a user interface associated with the medical provider; generating, based on the medical information, a response to the user-generated natural language query; and providing the response to the user interface associated with the medical provider.
Clause 8. The computer-implemented method, comprising: training, based on medical information from medical records of other patients, a model that defines associations between the medical information and the medical entities; applying the medical information of the patient to the model; receiving an indication, from the model, of the medical entities that the medical information is associated with; and generating, based on the medical entities, visual representations of the medical records.
Clause 9. The computer-implemented method, further comprising: generating the visual representation by: generating graphical user interface elements to represent the visual representations of the medical records over the period of time; and causing the graphical user interface elements to be presented on a single user interface generated by the cognitive intelligence platform.
Clause 10. A system, comprising: a memory device containing stored instructions; a processing device communicatively coupled to the memory device, wherein the processing device executes the stored instructions to: receive, from a plurality of electronic health record systems, medical records associated with medical encounters of a patient with one or more medical providers over a period of time, the medical records including medical information related to care of the patient; analyze the medical records by classifying the medical information included in the medical records into medical entities, the medical entities including categories having particular shared characteristics related to the care of the patient; generate, based on the medical entities, visual representations of the medical records over the period of time; and provide the visual representations of the medical records to a medical provider.
Clause 11. The system, wherein the processing device further executes the stored instructions to: redact personally identifiable information from the medical records.
Clause 12. The system, wherein the processing device further executes the stored instructions to: identify a negation cue modifying a piece of text in the medical information, the piece of text indicating an aspect of the care of the patient; and determine, based on the negation cue, a probability of existence of the aspect of care of the patient.
Clause 13. The system, wherein the processing device further executes the stored instructions to: classify one or more aspects of the medical information included in the medical records into any of the following: definite negations, definite preferences, definite rule outs, probable negations, probable preferences, and probable rule outs.
Clause 14. The system, wherein the medical entities includes at least one of the following: conditions, medications, laboratory results, test results, procedures, symptoms, and life style choices.
Clause 15. The system, wherein the processing device further executes the stored instructions to: eliminate, based on the medical information, one or more potential diagnoses from a list of possible conditions the patient may have.
Clause 16. The system, wherein the processing device further executes the stored instructions to: receive a user-generated natural language query from a user interface associated with the medical provider; generate, based on the medical information, a response to the user-generated natural language query; and provide the response to the user interface associated with the medical provider.
Clause 17. The system, wherein the processing device further executes the stored instructions to: train, based on medical information from medical records of other patients, a model that defines associations between the medical information and the medical entities; apply the medical information of the patient to the model; receive an indication, from the model, of the medical entities that the medical information is associated with; and generate, based on the medical entities, visual representations of the medical records.
Clause 18. A computer readable media storing instructions that are executable by a processor to cause a processing device to execute operations comprising: receive, from a plurality of electronic health record systems, medical records associated with medical encounters of a patient with one or more medical providers over a period of time, the medical records including medical information related to care of the patient; analyze the medical records by classifying the medical information included in the medical records into medical entities, the medical entities including categories having particular shared characteristics related to the care of the patient; generate, based on the medical entities, visual representations of the medical records over the period of time; and provide the visual representations of the medical records to a medical provider.
Clause 19. The computer-readable medium, wherein the processing device is further caused to execute operations comprising: redact personally identifiable information from the medical records.
Clause 20. The computer-readable medium, wherein the processing device is further caused to execute operations comprising: identify a negation cue modifying a piece of text in the medical information, the piece of text indicating an aspect of the care of the patient; and determine, based on the negation cue, a probability of existence of the aspect of care of the patient.

The foregoing description, for purposes of explanation, used specific nomenclature to provide a thorough understanding of the described embodiments. However, it should be apparent to one skilled in the art that the specific details are not required in order to practice the described embodiments. Thus, the foregoing descriptions of specific embodiments are presented for purposes of illustration and description. They are not intended to be exhaustive or to limit the described embodiments to the precise forms disclosed. It should be apparent to one of ordinary skill in the art that many modifications and variations are possible in view of the above teachings.

The above discussion is meant to be illustrative of the principles and various embodiments of the present invention. Numerous variations and modifications will become apparent to those skilled in the art once the above disclosure is fully appreciated. It is intended that the following claims be interpreted to embrace all such variations and modifications.

What is claimed is:

1. A computer-implemented method performed by a cognitive intelligence platform, the method comprising:
receiving, from a plurality of electronic health record systems, medical records associated with medical encounters of a patient with one or more medical providers over a period of time, the medical records including medical information related to care of the patient;
analyzing the medical records by classifying the medical information included in the medical records into medical entities, the medical entities including categories having particular shared characteristics related to the care of the patient;
generating, based on the medical entities, visual representations of the medical records over the period of time; and
providing the visual representations of the medical records to a medical provider.

2. The computer-implemented method of claim 1, the method further comprising:
redacting personally identifiable information from the medical records.

3. The computer-implemented method of claim 1, wherein analyzing the medical records further comprises:
identifying a negation cue modifying a piece of text in the medical information, the piece of text indicating an aspect of the care of the patient; and
determining, based on the negation cue, a probability of existence of the aspect of care of the patient.

4. The computer-implemented method of claim 3, wherein analyzing the medical records further comprises:
classifying one or more aspects of the medical information included in the medical records into any of the following: definite negations, definite preferences, definite rule outs, probable negations, probable preferences, and probable rule outs.

5. The computer-implemented method of claim 1, wherein the medical entities includes at least one of the following: conditions, medications, laboratory results, test results, procedures, symptoms, and life style choices.

6. The computer-implemented method of claim 1, the method further comprising:
eliminating, based on the medical information, one or more potential diagnoses from a list of possible conditions the patient may have.

7. The computer-implemented method of claim 1, the method further comprising:
receiving a user-generated natural language query from a user interface associated with the medical provider;
generating, based on the medical information, a response to the user-generated natural language query; and
providing the response to the user interface associated with the medical provider.

8. The computer-implemented method of claim 1, comprising:
training, based on medical information from medical records of other patients, a model that defines associations between the medical information and the medical entities;
applying the medical information of the patient to the model;
receiving an indication, from the model, of the medical entities that the medical information is associated with; and
generating, based on the medical entities, visual representations of the medical records.

9. The computer-implemented method of claim 1, further comprising:
generating the visual representation by:
generating graphical user interface elements to represent the visual representations of the medical records over the period of time; and
causing the graphical user interface elements to be presented on a single user interface generated by the cognitive intelligence platform.

10. A system, comprising:
a memory device containing stored instructions;
a processing device communicatively coupled to the memory device, wherein the processing device executes the stored instructions to:
receive, from a plurality of electronic health record systems, medical records associated with medical encounters of a patient with one or more medical providers over a period of time, the medical records including medical information related to care of the patient;
analyze the medical records by classifying the medical information included in the medical records into medical entities, the medical entities including categories having particular shared characteristics related to the care of the patient;
generate, based on the medical entities, visual representations of the medical records over the period of time; and
provide the visual representations of the medical records to a medical provider.

11. The system of claim 10, wherein the processing device further executes the stored instructions to:
redact personally identifiable information from the medical records.

12. The system of claim 10, wherein the processing device further executes the stored instructions to:
Identify a negation cue modifying a piece of text in the medical information, the piece of text indicating an aspect of the care of the patient; and
determine, based on the negation cue, a probability of existence of the aspect of care of the patient.

13. The system of claim 10, wherein the processing device further executes the stored instructions to:
classify one or more aspects of the medical information included in the medical records into any of the following: definite negations, definite preferences, definite rule outs, probable negations, probable preferences, and probable rule outs.

14. The system of claim 10, wherein the medical entities includes at least one of the following: conditions, medications, laboratory results, test results, procedures, symptoms, and life style choices.

15. The system of claim 10, wherein the processing device further executes the stored instructions to:
eliminate, based on the medical information, one or more potential diagnoses from a list of possible conditions the patient may have.

16. The system of claim 10, wherein the processing device further executes the stored instructions to:
receive a user-generated natural language query from a user interface associated with the medical provider;
generate, based on the medical information, a response to the user-generated natural language query; and
provide the response to the user interface associated with the medical provider.

17. The system of claim 10, wherein the processing device further executes the stored instructions to:
train, based on medical information from medical records of other patients, a model that defines associations between the medical information and the medical entities;
apply the medical information of the patient to the model;
receive an indication, from the model, of the medical entities that the medical information is associated with; and
generate, based on the medical entities, visual representations of the medical records.

18. A computer readable media storing instructions that are executable by a processor to cause a processing device to execute operations comprising:
receive, from a plurality of electronic health record systems, medical records associated with medical encounters of a patient with one or more medical providers over a period of time, the medical records including medical information related to care of the patient;
analyze the medical records by classifying the medical information included in the medical records into medical entities, the medical entities including categories having particular shared characteristics related to the care of the patient;
generate, based on the medical entities, visual representations of the medical records over the period of time; and
provide the visual representations of the medical records to a medical provider.

19. The computer-readable medium of claim 18, wherein the processing device is further caused to execute operations comprising:
redact personally identifiable information from the medical records.

20. The computer-readable medium of claim 18, wherein the processing device is further caused to execute operations comprising:
identify a negation cue modifying a piece of text in the medical information, the piece of text indicating an aspect of the care of the patient; and
determine, based on the negation cue, a probability of existence of the aspect of care of the patient.
